(19) United States
(12) Patent Application Publication
(10) Pub. No.: US 2010/0226902 A1
(43) Pub. Date: Sep. 9, 2010

(54) Method For Treating Wounds With Enriched Platelet Wound Healant

(75) Inventor: Carelyn P. Fylling, Walker, MN (US)

Correspondence Address: FITCH EVEN TABIN & FLANNERY, 120 SOUTH LASALLE STREET, SUITE 1600, CHICAGO, IL, 60603-3406, US

(73) Assignee: CYTOMEDIX, INC., Rockville, MD (US)

(21) Appl. No.: 11/993,188

(22) PCT Filed: Jun. 30, 2006
(86) PCT No.: PCT/US06/25962

§ 371 Date: Dec. 19, 2007

Related U.S. Application Data
(60) Provisional application No. 60/695,253, filed on Jun. 30, 2005.

(51) Int. Cl. A61K 35/16; A61P 17/02; B04B 5/02

(52) U.S. Cl. 424/93.72; 494/16

(57) ABSTRACT

An improved method for treating wounds with an enriched platelet composition is provided in which a small amount of a patient's blood is used to prepare, at point-of-care, a platelet-rich plasma (PRP) from which a gel is produced. The method involves the use of improved materials to improve the ease and speed at which wounds may be treated at the point-of-care.

### CROSS-REFERENCE

This application claims the benefit of Provisional Application Ser. No. 60/695,253 filed on Jun. 30, 2005, the disclosure of which is hereby incorporated in its entirety by reference.

### FIELD OF THE INVENTION

The invention relates to an improved method for treating wounds with an enriched platelet composition.

### BACKGROUND OF THE INVENTION

There have been many different substances and methods developed in the past for treating wounds, depending upon the type and location and severity of the wound. A wound is generally defined as an injury to an area of the body of a human or animal. Although injury to the surface of the skin is the most well known type of wound, the surfaces of internal organs may also be wounded, such as during surgery, rupture of the spleen or liver, or resulting from traumatic blows to the body surface in the vicinity of an internal organ.

Medical practice characterizes wounds as chronic or acute, according to the persistence and severity of the wound. A chronic wound is one that is prolonged or lingering, rather than promptly healed. An acute wound is one that occurs relatively quickly, and heals relatively quickly as well. Tissue wounds may have a wide spectrum of manifestations, as small as merely an abnormal microscopic tear or fissure in tissue (or a surface thereof), or as large as the abrasion or ablation of the skin covering a substantial portion of the body, such as in a burn victim. Acute wounds covering a large or movable surface are usually the most difficult to guard from infection, and to heal.

In particular, diabetic foot ulcers are a common cause of amputation. More that 20.8 million persons in the U.S. have diabetes mellitus. 2002 data estimates from the Centers for Disease Control and Prevention indicate that 82,000 lower limb amputations were performed with persons with diabetes. (Center for Disease Control and Prevention 2005.) Characteristic pathological changes attributed to autonomic and sensory neuropathy, often combined with vascular disease, lead to a high-risk situation for the person with diabetes. (American Diabetes Association 1999; Mani et al. 1999.) Persons who have had such pathology and experience trauma or infection are at a high risk for developing ulceration of the foot or ankle. One study has found that in 81% of cases faulty wound healing contributed to amputation. (Pecoraro et al. 1990.) Thus, a need exists for wound treatments that can reduce the rate of faulty wound healing and prevent amputations.

According to the American Diabetes Association, more than 60% of non traumatic lower-limb amputations occur in people with diabetes; the rate of amputation for people with diabetes is 10 times higher than for people without diabetes; Mexican Americans are 1.8 times as likely, non-Hispanic Blacks are 2.7 times as likely, and American Indians are three to four times as likely to experience lower-limb amputations. Amputation rates are 1.4 to 2.7 times higher in men than women with diabetes. One study estimates 67,000 diabetes-related lower extremity amputations (LEA) and a similar study indicates that LEA have resulted in a total of 984,000 hospital days, each length of stay averaging 15 days. (Frykberg et al. 2000.) Nonhealing diabetic foot ulcers and the resulting potential amputations present significant costs to the healthcare system and reduce patient quality of life.

The goal of diabetic foot ulcer treatment is to obtain wound closure as expeditiously as possible. Accepted therapeutic objectives and standards of care for diabetic foot ulcers include wound debridement, pressure relief in the wound area, appropriate wound management (for example, moist wound healing), infection management, ischemia management, medical management of comorbidities, and surgical management as needed. (Frykberg et al. 2000.) Emerging cellular therapies such as platelet-rich plasma (PRP) can have an adjunctive role in a standardized, quality treatment plan.

Blood and bodily fluids include various substances that affect wound healing. The blood is the primary medium for delivering healing agents to the wound site and for transporting foreign or harmful substances away from the wound. Whole blood is primarily comprised of three main types of cells suspended in a protein rich solution known as plasma. The three main cell types in whole blood are erythrocytes (a.k.a. red blood cells), leukocytes (a.k.a. white blood cells) and thrombocytes (a.k.a. platelets). The red blood cells are the iron-containing cells that facilitate the transport and transfer of oxygen to body tissue, and the removal of carbon dioxide. The white blood cells perform a variety of functions such as phagocytosis of foreign bodies and production of antibodies, and are primarily responsible for fighting infection and foreign substances within the blood or wound site. Platelets perform many functions such as plugging leaks in blood vessels and helping begin the process leading to the formation of a blood clot. Platelets contain substances known as growth factors that facilitate the formation of new tissue.

Platelet releasates, including multiple growth factors, have been used to treat wounds since 1985. In vivo prospective controlled studies as well as retrospective and cost effectiveness studies documenting the effect of this therapy have been published. (Holloway et al. 1993; Steed et al. 1992; Atri et al. 1990; Knighton et al. 1990a,b; Fylling et al. 2001; Glover et al. 1997; Keyser 1993; Fylling 1992; Fylling et al. 1990a,b; Doucette et al. 1989; Knighton et al. 1989; Knighton et al. 1986; Bentkover et al. 1993.) In vitro research has shown that platelets contain components and properties for wound healing. (Bennet et al. 1993.) Likewise, plasma contains fibrin matrix. (Mossesson 2005.) One of the proteins suspended in plasma is fibrinogen, which reacts with substances, such as thrombin, released into (or attracted by) wound sites to produce sticky strands of fibrin. Such reactions result in the cross linking of the fibrin strands to form a mesh, or matrix, that holds and supports the deposit or growth of other tissue materials at the wound site.

Although there are several methods for separating whole blood into its various components, one of the most convenient and expeditious methods is accomplished by differentially centrifuging blood or some of its components (i.e., apheresis). Using apheresis, the red and white blood cells and plasma may be separated out and returned to the donor's or patient's body, leaving the sequestered platelets in essentially concentrated form for use in wound healing techniques. From blood extracted from a patient, the platelets may thus be obtained and activated for use on the same patient. Methods of using a patient's own blood are called “autologous” or “autogenic” donor methods. Methods using blood donated by one or more third parties for use by a patient are called “homologous” or “heterologous” donor methods, or collectively called “allogenic” methods.

The wound healing process is generally considered to occur in several stages, generally known as the healing cascade. After tissue injury, platelets are among the first cells to appear in the vicinity of the wound. Activation of a platelet by an agonist such as thrombin, leads to the release of granule material from within the platelet. Such granulation activation results in the release of proteins known as growth factors, primarily concentrated in the alpha granules of platelets. Growth factors act as mitogens and chemoattractants to direct cellular growth and migration, thus stimulating the formation of new tissue. When applied to wounds, growth factors have been known to increase the rate of collagen deposition, vascular ingrowth, fibroblast proliferation and overall healing. The release of a protein known as platelet-derived growth factor (PDGF) is a chemotactic signal for monocytes, neutrophils, and fibroblasts which then move into the wound, to begin the inflammatory stage of the healing process. During this time, monocytes secrete a number of factors including PDGF and transforming growth factor-β1 (also found in platelets), which recruits and activates fibroblasts, to begin the repair stage of the healing process. Subsequently, wound healing continues through the process of collagen remodeling within the wound.

Knighton pioneered the preparation and use of wound treatment compositions derived from platelet enriched concentrates as disclosed in U.S. Pat. No. 5,165,938; Worden U.S. Pat. No. 6,524,568; Worden U.S. Pat. No. 6,303,112; Hood U.S. Pat. No. 5,733,545; and Gordiner U.S. Pat. No. 5,599,558 also relate to platelet derived wound treating compositions.

Platelet concentrates are typically isolated by the process of differential centrifugation which essentially allows separating the patient's own blood into at least three different components: packed erythrocytes (red blood cells), plasma and platelet concentrate. Platelet-rich plasma can be combined with a solution of either sodium or calcium mixed with thrombin (“calcified thrombin”), which instantaneously forms a composition of activated platelets that can be utilized as a wound treatment.

The use of PRP in the surgical setting; notably, the orthopedic, plastic surgery, and dental fields is known. (Kassolis et al. 2005; Grageda 2004; Weilbrich et al. 2004.) However, current PRP separation systems require specialized technicians to perform the necessary procedures. Thus, it would be desirable to provide an autologous PRP separation system to heal wounds that can be used by health professionals within a traditional healthcare setting. Furthermore, there is an ongoing need to improve the ease and speed at which wounds may be treated at the point-of-care.

### SUMMARY OF THE INVENTION

The present invention provides an improved system for treating wounds with an enriched platelet composition. The system of the present invention uses a small amount of a patient's blood to prepare, at point-of-care, platelet-rich plasma (PRP) from which a gel is produced. One aspect of the invention involves the use of improved materials to improve the ease and speed at which wounds may be treated at the point-of-care. A small, compact, point-of-care system is provided which makes this technology available to multiple care providers, including physician offices, hospital units, outpatient clinics, long-term care facilities, and home health care staff. All of the components and reagents utilized are compatible for human use.

### BRIEF DESCRIPTION OF THE DRAWINGS

FIG. 1 is a flow chart for a method of preparing and applying an enriched platelet composition at a point-of-care;

FIG. 2 is a flow chart of the trial profile described in Example 1;

FIG. 3 is a graph showing the per protocol patient group (n=40) Kaplan-Meier time-to-healing curves; and

FIG. 4 is a graph showing the majority wound patient group (n=35) Kaplan-Meier time-to-healing curves.

### DETAILED DESCRIPTION

Before the present invention is described in detail, it is to be understood that the invention is not limited to the particular configurations, process steps, and materials expressly disclosed herein. The invention includes those that are implicit or inherent in the disclosures set forth herein, and all equivalents of any element or limitation thereof. The scope of the present invention is limited only by the claims and equivalents thereof.

The phrase blood collecting or blood extraction (or similar phrase) includes techniques known in the field for obtaining a volume of blood from a patient or other donor, such as (for example) inclusion of anticoagulation materials, the use of blood drawing and infusion apparatus.

The term thrombin may include calcified thrombin, in particular, about 5,000 units of thrombin per 5 ml of 10% of aqueous calcium chloride solution; it may include calcified bovine thrombin as well as autologous thrombin, allogeneic thrombin or recombinant human thrombin.

The term anti-oxidant refers to any material(s) having anti-oxidant properties. Anti-oxidant would include, without limitation, vitamins such as vitamins A, C, and E and non-vitamins such as β-carotene.

The conjunctive “and” may also be taken to include the disjunctive “or”, and vice versa, whenever necessary to give the claims of this patent application the broadest interpretation and construction possible. Likewise, when the plural form is used it may be taken to include the singular form and vice versa.

The present invention uses a small amount of the patient's blood to prepare at point-of-care platelet-rich plasma (PRP) from which a gel is produced and applied to a wound bed of the patient. The following sets forth the details of the procedure.

### Blood Collection

Using a butterfly phlebotomy set, approximately 5-30 cc, and preferably 20 cc, of whole blood is drawn from the patient according to usual and customary procedures. More or less blood may be drawn depending on the size of the wound to be treated.

The blood is collected into one or more 5 mL vacuum blood collection tubes. The preferred blood collection tubes are tubes which have been tested and approved for human use, such as the Custom Made Vacuum Blood Collection Tubes for the AutoloGel™ System, which include USP Grade Anticoagulant Citrate Dextrose (ACD). To be approved for human use, blood collection tubes must be shown to be latex-free, non-leaching, and non-cytotoxic. The Custom Made Vacuum Blood Collection Tubes for the AutoloGel™ System have been so tested and shown to be suitable for human use.

Advantageously, the Custom Made Vacuum Blood Collection Tubes for the AutoloGel™ System can be both used to withdraw the patient's blood and placed directly into the centrifuge. This results in improved speed and ease of use over the prior art, in which the patient's blood had to first be drawn into a syringe and subsequently transferred into a centrifuge tube.

### Centrifuge Process

Next, the whole blood is centrifuged and separated into red blood cells (RBC), white blood cells (WBC), and platelet-rich plasma (PRP). The preferred centrifuge for use in the present invention consists of a table-top, non-self decanting, rotor spin centrifuge and rotor sleeve disposables designed to allow for rapid automatic separation of plasma and red blood cells from a small volume of whole blood. It spins at a maximum speed of at least about 8300 rpm producing a maximum force of at least about 4236 g. Thus, it is relatively small, portable, and fast. One suitable centrifuge is the known as the StatSpin® 30 Primary Tube Centrifuge, Model No. M503-22, manufactured by IRIS Sample Processing (formerly, StatSpin, Inc.). Other suitable centrifuges may be used as well.

The top of each vacuum tube should be cleansed prior to placing in the centrifuge. The tubes are placed into the centrifuge and, if there are an odd number of tubes, a tube of water is added for balance. The centrifuge cover is closed and the centrifuge is activated. Preferably, the unit will run for about 1.5 minutes and provide a full separation of platelet-rich plasma (PRP) and red blood cells. The unit will also preferably shut off automatically.

### Gelling Process

Next, the liquid PRP is drawn into a syringe, and the reagents (thrombin, calcium chloride, and ascorbic acid, for example) are added to the PRP in predetermined formulations, concentrations, and sequence, to form a gel. In one form, the gel is preferably made from 8 ml PRP to which 1 mL aliquots of calcified thrombin mixture (5 cc of 10% calcium chloride mixed into 5000 U.S. units of bovine thrombin) and ascorbic acid are added.

Thus, in one preferred form, a 20 cc syringe, blunt needle, and three-way stopcock (mixing chamber) are assembled. After centrifugation, the elastomer tops of the vacuum blood collection tubes are removed and the PRP is extracted using the syringe assembly. Preferably, about 8 ml of PRP is extracted; however, more or less PRP may be used as needed. The resulting concentration of platelets is preferably the optimum concentration for efficacy.

Next, the desired amount of ascorbic acid and calcified thrombin mixture are added to the syringe assembly through the mixing chamber. Preferably, about 1 mL of ascorbic acid and about 1 mL of the calcified thrombin mixture are added, however, more or less ascorbic acid and calcified thrombin mixture may be added as needed depending on the amount of PRP used.

The desired amount of ascorbic acid is added to the syringe assembly through the mixing chamber. The syringe assembly is subsequently inverted once to mix. The calcified thrombin mixture also added to the syringe assembly through the mixing chamber. Then, the syringe assembly is inverted several times to mix until the desired gel consistency is obtained.

The materials used in the above-described gelling process are all preferably tested and approved for human use.

### Wound Treatment

When the mixture in the syringe assembly appears to be gelled (usually about 10-30 seconds), the mixture is immediately applied to the prepared wound. If desired, the addition of the calcified thrombin mixture may be delayed so as to delay the gelling of the mixture. A contact layer interface dressing is preferably placed directly over the gel on the wound. An outer primary dressing (and secondary dressing, if needed) is preferably also applied to cover the wound and keep it moist.

The gel, including the autologous blood-derived components promotes healing of the wound by interacting directly or indirectly with body tissues. In addition, it maintains a desirably moist wound environment. The healing effect results from the blood-derived components and properties of the PRP gel that may advantageously be formed from a small amount of blood drawn from the patient-at-point of care.

The following examples describe and illustrate the processes and products of the present invention. These examples are intended to be merely illustrative of the present invention, and not limiting thereof in either scope or spirit. Those skilled in the art will readily understand that variations of the materials, conditions, and processes described in these examples can be used. All references cited herein are incorporated by reference in their entirety.

### Example 1

The following example demonstrates the safety and effectiveness of treating diabetic foot ulcers with the PRP gel obtained according to the present invention versus a control treatment (normal saline gel). (Driver et al. 2006.) The primary objective of the 12-week study was to compare the safety and incidence of complete wound closure between PRP gel- and control-treated wounds at the end of the study. Secondary objectives included comparing the rate of wound healing during the 12-week study and incidence of wound recidivism among healed ulcers during a 3-month follow-up period. Safety variables included adverse events, serious adverse events, and clinical laboratory tests.

### Study Design and Methods

This prospective, randomized, controlled, double blinded, multicenter trial was conducted under the U.S. Food and Drug Administration (FDA) Investigational Device Exemption (IDE) regulations. Constella Clinical Informatics (Durham, N.C.) served as the Clinical Research Organization (CRO) to implement and monitor the trial, gather the data into a central database, and audit the data. A data safety monitoring board provided evaluations of the safety-related events throughout the treatment phase of the study. In dependent statisticians were contracted to initially power the study, develop the statistical plan, and analyze the safety and effectiveness data.

Fourteen (14) investigative sites from across the country participated in the study. Sites included wound care physicians' and podiatrists' offices, outpatient wound care centers, a university-based college of podiatric medicine clinic, Veteran's Administration wound care clinics, and an Army hospital limb preservation program. Each site obtained IRB approval to conduct the study.

Study eligibility. Persons with type 1 or type 2 diabetes between the ages of 18 and 95 with an ulcer of at least 4 weeks' duration were eligible for the study if they met additional inclusion/exclusion criteria: hemoglobin A1C<12; index foot ulcer located on the plantar, medial, or lateral aspect of the foot (including all toe surfaces); and wound area (length×width) measurement between 0.5 cm² and 20 cm², inclusive. Wounds located under a Charcot deformity had to be free of acute changes and must have under gone appropriate structural consolidation. The index ulcer had to be clinically noninfected (although a culture was obtained, infection was diagnosed through clinical signs and symptoms rather than culture results (Lipsky et al. 2004)) and full-thickness without exposure of bone, muscle, ligaments, or tendons (University of Texas Treatment-Based Diabetic Foot Classification System: Grade 1 A (Armstrong et al. 1998)).

The protocol required that post debridement the ulcer would be free of necrotic debris, foreign bodies, sinus tracts, tunneling, and undermining; comprised of healthy vascularized tissue; and at least 4 cm from any additional wound. Additionally, the limb had to have adequate perfusion as shown by examination and non-invasive vascular testing ankle brachial index (ABI) and toe brachial index (TBI). Women of child bearing age could not be pregnant or lactating; both men and women had to be willing to use a medically accepted form of birth control throughout the trial and for 6 months following. Patient history, physical examination (including a Semmes Weinstein monofilament test for neuropathy), and blood for baseline laboratory studies were obtained.

Approved, informed, signed consent stipulating that the patient was able to comply with all specified care and visit requirements was secured from the patient, caregiver, or legal representative before study enrollment. The Investigator documented reasonable expectation that the patient was medically stable and capable of completing the study. Study exclusion criteria are listed in Table 1.

TABLE 1
STUDY EXCLUSION CRITERIA

Patient currently enrolled in another investigational device or drug trial or previously enrolled
(within last 30 days) in investigative research of a device or pharmaceutical agent
Ulcer decreased ≧50% in area during 7-day screening period
Ulcer is due to non-diabetic etiology
Patient's blood vessels are non-compressible for ABI testing
Evidence of gangrene in ulcer or on any part of the foot
Patient has radiographic evidence consistent with diagnosis of acute Charcot foot
Patient is currently receiving or has received radiation or chemotherapy within 3 months of
randomization
Topical, oral, or IV antibiotic/antimicrobial agents or medications have been used within 2
days (48 hours) of randomization
Patient has received growth factor therapy (e.g., autologous platelet-rich plasma gel,
becaplermin, bilayered cell therapy, dermal substitute, extracellular matrix) within 7 days of
randomization.
Screening serum albumin level <2.5 g/dL
Screening hemoglobin <10.5 mg/dL
Screening platelet count <100 × 109/L
Patient is undergoing renal dialysis, has known immune insufficiency, known abnormal
platelet activation disorders - i.e., gray platelet syndrome, liver disease, active cancer (except
remote basal cell of the skin), eating/nutritional, hematologic, collagen vascular disease,
rheumatic disease, or bleeding disorders
History of peripheral vascular repair within the 30 days of randomization
Patient has known or suspected osteomyelitis
Surgical correction (other than debridement) required for ulcer to heal
Index ulcer has exposed tendons, ligaments, muscle, or bone
Patient is known to have a psychological, developmental, physical, emotional, or social
disorder, or any other situation that may interfere with compliance with study requirements
and/or healing of the ulcer
History of alcohol or drug abuse within the last year prior to randomization
Patient has inadequate venous access for blood draw
Patient has a religious or cultural conflict with the use of platelet gel treatment

After meeting all initial inclusion criteria and signing the informed consent, all patients completed a 7-day screening period. This included initial excision/debridement, baseline wound measurements and evaluation, and application of the control saline gel to the wound. For standardization, sharp debridement guidelines were provided as part of the protocol. Patients, a family member, or other designated parties were provided supplies and instructed to change the dressings once midway through the screening period. The patient also was required to use a fixed ankle-foot orthoses that could be removed for the dressing change and at night. Crutches or a walker were used for added safety. Screening data were captured on case report forms (CRFs) for data analysis. Patients whose wounds reduced in area by >50% during the screening period were not randomized to treatment and discontinued from any further study participation because they appeared to be able to heal without more advanced intervention.

Randomization and blinding procedures. The randomization schedule was electronically generated, blocked per investigational center, and provided to the site by the contract research organization (CRO). Each eligible study participant was assigned to one of two treatment groups, PRP or control, and received the next available consecutive randomization number. Each site had one designated “unblinded” person to treat the patient (also blinded) and maintain documents in a secure private area to maintain blinding of the investigator, investigative site staff, patient, sponsor, and CRO staff and monitor. This person did not participate in any other aspect of the patient's care. The blinded investigators and staff measured the wounds; performed all tests, assessments, and debridement; and determined wound closure. A strategically placed drape prohibited the patient from seeing which treatment was applied to the wound. Blood was drawn from both the treatment and control patients to maintain blinding.

PRP preparation process. As described above, the PRP separation system of the present invention and utilized in the study is a new generation, point-of-care system for processed autologous platelets and plasma to be used for the treatment of non-healing wounds. This system comprises a small portable centrifuge to separate whole blood into PRP and a convenience kit that includes items for the blood draw, processing, and PRP gel application. The platelet-rich plasma gel (AutoloGel™, Cytomedix, Inc. Rockville, Md.) was used to treat patients in the treatment group. Wounds in the control group were treated with saline gel (Normlgel®, Molnlycke Health Care, Norcross, Ga.). Either PRP gel or saline gel was applied to the prepared wound bed.

The first step of the PRP separation process included performing a venipuncture to draw <20 mL of blood, depending on the wound size, from the patient. The blood was spun in a small, portable centrifuge for 1.5 minutes to separate the PRP from the whole blood. The PRP was extracted into a syringe where reagents were added to activate the platelets and plasma as well as to achieve proper gel consistency (gel consistency was usually attained within 15 to 30 seconds). The gel then was immediately applied to the wound. A contact layer dressing was applied over the gel. A foam dressing (non-absorbent side) was placed over the contact dressing layer so the PRP gel was not absorbed. This was covered with the absorbent side of a foam dressing (to absorb any leaking wound exudates) and secured. For protection, barrier cream was placed on intact skin surrounding the wound.

For patients randomized to the control group, normal saline gel was applied to the wound following wound bed preparation. Similar to PRP gel application, a contact layer dressing was applied over the saline gel, followed by the non-absorbent side of a foam dressing, and covered with the absorbent side of a foam dressing before being secured.

Clinical evaluations and procedures. Wounds were assessed and measured (length, width, and depth using a metric tape measure at each visit. The measurements and other wound variables including undermining or tunneling, characteristics of wound exudates (i.e., presence, color, amount, and odor), necrotic tissue, and granulation tissue were documented. (Cooper 2000.)

Care and management efforts provided at each treatment visit included cleansing and assessing the wound and obtaining vital signs and an interim wound history, including information regarding adverse events, concomitant medications, nutrition and weight-bearing status, and other aspects of care since the last visit. A facility designee performed phlebotomy; the unblended person performed all subsequent gel processing. The principal investigator, who did not observe treatment procedures, directed wound care provision during the care visit.

The need for consistency in product application and maintenance of the blinding process dictated that dressings were applied only at the Investigator's site except for the provision of a one-time dressing change at home should circumstances prevent clinic attendance. Patients returned twice weekly at 3- or 4-day intervals; procedures and processes described were performed at each visit for a maximum of 12 weeks. Treatment continued until the wound healed, the 12-week treatment phase was complete, or patient study participation was terminated by the Investigator, sponsor, or because the patient withdrew consent or failed to return for visits.

To evaluate safety, clinical laboratory tests were conducted through out the study to determine the impact of treatment interventions (see Table 2). Expected or unexpected adverse events (AE) that occurred during the course of the study, whether observed by the Investigator or by the patient, were reported in detail. The Investigator monitored the patient for AEs or lab abnormalities until the parameter returned to normal or it was determined that follow-up was no longer necessary.

TABLE 2
LABORATORY STUDIES' SCHEDULE

| | | q2 Weeks | | | |
|---|---|---|---|---|---|
| | Baseline/ | During | | | |
| Laboratory Parameter | Screening | Treatment | 6 Weeks | 12 Weeks | 24 Weeks |
| Complete blood count | X | X | X | X | |
| Chemistry 7 panel | X | As needed | X | X | |
| serum albumin | | | | | |
| HgbaA1C | X | | | | X |
| Partial thromboplastin | X | X | X (if during | X (if during | |
| time, prothrombin | | | treatment) | treatment) | |
| time, thrombin time | | | | | |
| Antibody for Factor V | X | | X | X | X |
| Fibrinogen | X | | | | |
| Antigenic fibrinogen | PRN positive | | | | |
| | fibrinogen | | | | |

End-of-treatment visit procedures. When the Investigator pronounced the wound closed (i.e., 100% epithelialized) the patient was scheduled for a visit 1 week later but asked to continue wearing the offloading orthosis walker. At this visit, if the wound had reopened, the patient was re-entered into the study at the same timeline (coinciding with the return visit for continued care) and continued until the wound either healed or until week 13, visit 1 without healing. If the wound stayed healed after the 1-week interval, the patient entered the follow-up phase and returned after 3, 7, and 11 weeks.

During this 3-month follow-up phase, the healed wound was evaluated for break down and the patient was queried regarding adverse events.

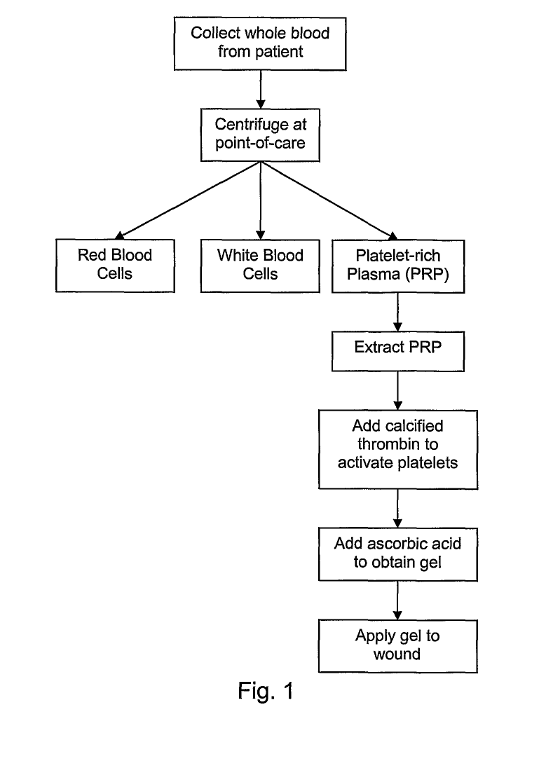

At the end of the 12-week treatment period, unhealed wounds were treated per physician protocol. The patient was discharged from the site for follow-up at a facility of his/her choice. All participants were asked to return for final Factor V testing at week 24 post-randomization date. End-of-study occurred at completion of the week 24 clinical lab evaluation, withdrawal of patient consent, or death of the patient. Patient procedures/instructions were repeated, including a complete history and physical examination with testing of pedal pulses, vascular testing (ABI and TBI), Semmes-Weinstein monofilament testing, laboratory tests, dressing change as per the Investigator's order, and education with a discussion of healthcare options (see FIG. 1).

### Statistical Analysis

Healing rate. The power of the study was determined based on two data sources. The PRP gel healing rate was determined from the healing rate in an unpublished diabetic foot ulcer retrospective study (Fylling et al. 2001) and feed back from clinicians who had used the PRP gel. (Beriou et al. 2004.) The control group healing rate was based on a meta-analysis of healing rates in the control groups of 10 prospective studies. (Margolis et al. 1999.)

Sample size. To determine the sample size, the expected proportion of patients with completely closed wounds was determined to be 0.60 (nt) in the PRP gel arm and 0.20 (π_{c}) in the control arm. To calculate the sample size, the following null hypothesis (H₀) and the alternative hypothesis (H_{A}) were formulated:

H₀: πₜ=π_{c} versus H₀: πₜ≠π_{c}.

A Fisher's exact test with a 0.050, two-sided significance level would have 80% power to detect the different between PRP gel proportion, πₜ, and control proportion, π_{c}, of 0.20 when the sample size in each arm of treatment is 27 (i.e., a total of 54 patients). The sample size was increased from 54 to 72 patients to accommodate drop-outs.

The primary efficacy variable was the proportion of patients with a healed wound. Fisher's exact test was applied to compare the two treatments for proportions healed within each group of investigative sites and all groups combined.

Because of varying enrollment at each site (between one and 14 patients), sites were grouped for analysis purposes according to provider setting and demographics (Groups I to V). Due to the varying nature of investigative sites and their ability to enroll patients, sites were grouped into five categories: teaching facilities, army facility, physicians in private practice (two sites), and ambulatory care clinics. The goal was to enroll eight to 21 patients in each group. Results from the independent audit eliminated all patients in one group, leaving four groups for per protocol (PP) analysis.

Odds ratio/confidence level. The odds ratio and 95% confidence interval for each group were calculated for the proportion of patients with healed wounds. The Mantel-Haenszel (M-H) combined odds ratio (combining over groups) along with the 95% confidence interval was calculated. In addition, the M-H test for homogeneity of odds ratios of groups was performed. The M-H method was used to test the hypothysis whether the M-H combined odds ratio was one.

Additional variables. Other efficacy variables were 1) percent change in wound area at end-of-study visit (EOSV) from baseline (BL); 2) percent change in wound volume at EOSV from BL; 3) area closure rate per day at EOSV; and 4) volume closure rate per day at EOSV. These efficacy variables were of continuous type. For each of the variables, the two treatments were compared using Student's t-test. The statistical tests were performed using software package STATA, Release 8.2 (Stata Corporation, College Station, Tex.).

Kaplan-Meier. In addition, the Kaplan-Meier (Lee 1980; Kalbfleisch et al. 1980), product-limit method was used to analyze time to healing of the PP wounds and the majority of wound sizes dataset. Kaplan-Meier functions or curves of the PRP gel and control groups were obtained for each dataset. The log-rank test was used to test the hypothesis that the Kaplan-Meier healing functions are the same across the two treatments.

Laboratory safety. To evaluate clinical laboratory safety, observed values at each visit and changes from baseline at post-baseline visits and end point were summarized descriptively (number (n), mean, standard deviation (SD), minimum, median, maximum) for each treatment group. Results between treatments at the end point were compared using non-parametric analysis of variance techniques (Wilcoxon Rank Sum Test, two-sided).

Shift analyses. The number and percent of patients from each treatment group that shifted in and out of normal range from baseline to end point were calculated for each laboratory variable. To compare treatments for important shift changes, the number of patients whose laboratory results shifted from NORMAL or LOW at baseline to HIGH at end point and those shifting from NORMAL or HIGH at baseline to LOW at end point were compared in separate 2×2 tables and differences were tested for statistical significance using two-sided Fisher's exact tests. Patients whose laboratory results did not shift from baseline range or who were NORMAL at end point were analyzed together with those that had out of range shifts via exact tests to compare treatment groups (Exact test for row [R]×column [C] tables). With these three separate analyses, variables can be evaluated either for single direction shifts of interest or shifts in either direction, when both HIGH and LOW deviations may be of significance.

### Results

Initially, 129 patients provided Informed Consent forms and participated in active screening (see FIG. 1). Of these patients, 57 (44%) were dropped from the study due to reduction in the wound size of 50% during the 7-day screening period or for failure to meet the inclusion/exclusion criteria. Ultimately, 72 patients were enrolled, each patient having one wound (index ulcer) designated for study inclusion.

In the intent-to-treat (ITT) population, the mean and standard deviations (SD) for age, HgbA1C, wound area, and volume in the two treatments were not significantly different, but the wound volume in the PRP gel group was significantly more variable than in the control group (SDs 4.1 versus 1.2, P<0.0001) (see Table 3). Ulcer location information was missing for nine patients (three in PRP gel group and six in the control group). No significant differences in patient demographics, wound distribution, or ulcer location were observed between the two treatment groups. For purposes of the ITT analyses, the ITT population comprised all active patients who completed the study as well as those who were lost to follow-up, failed to complete the treatment, or had protocol violations. In the ITT group, 13 out of 40 patients (32.5%) in the PRP gel and nine out of 32 patients (28.1%) in the control group had completely healed wounds after 12 weeks (P=0.79). Because the results of the ITT analyses did not seem to reflect previous clinical outcomes, the study sponsor commissioned an independent audit to ensure study compliance with Good Clinical Practices (GCP) at the investigative sites.

During the audit, patient source documents, Case Report Forms (CRF), and other study source documents were reviewed. Five objective criteria were developed against which all audited patient records were evaluated. The protocol violations that caused exclusion of patients included use of the wrong centrifuge (causing the patient not to receive the right treatment (i.e., PRP gel); lack of source documentation to support case report form entries; and inclusion of patients and/or wounds that did not meet the inclusion/exclusion criteria. The predetermined statistical plan identified that analysis would be performed on patients who completed treatment; thus, patients with early termination due to reasons unrelated to the index wound and patients lost to follow-up also were excluded from the PP analysis.

TABLE 3
INTENT TO TREAT GROUP: PATIENT DEMOGRAPHIC AND
BASELINE WOUND VARIABLES

| | PRP Gel | | | | | Control | | | | | |
|---|---|---|---|---|---|---|---|---|---|---|---|
| | (n = 19) | | | | | (n = 21) | | | | | P |
| Variable | N | Mean | SD | Min | Max | N | Mean | SD | Min | Max | value |

Patient

| | | | | | | | | | | | |
|---|---|---|---|---|---|---|---|---|---|---|---|
| Age | 40 | 56.4 | 10.2 | 31.0 | 75.0 | 32 | 57.5 | 9.1 | 45.0 | 86.0 | NS* |
| (years) | | | | | | | | | | | |
| Hgb A₁C | 37 | 8.1 | 1.8 | 5.5 | 13.1 | 30 | 8.0 | 1.8 | 5.0 | 11.5 | NS |

Wound

| | | | | | | | | | | | |
|---|---|---|---|---|---|---|---|---|---|---|---|
| Area (cm²) | 40 | 4.0 | 5.3 | 4.0 | 24.0 | 32 | 3.2 | 3.5 | 0.5 | 15.8 | NS |
| Volume | 40 | 1.7 | 4.1 | 0.1 | 24.8 | 32 | 0.9 | 1.2 | 0.1 | 5.4 | NS |
| (cm³) | | | | | | | | | | | |

| | Treatment | | | | | |
|---|---|---|---|---|---|---|
| | PRP Gel (n = 40)† | | Control (n = 32)† | | Total | |
| | No. of | | No. of | | No. of | |
| Characteristics | patients | Percent | patients | Percent | patients | Percent |

Sex

| | | | | | | |
|---|---|---|---|---|---|---|
| Male | 32 | 80.00 | 27 | 84.38 | 59 | 81.94 |
| Female | 8 | 20.00 | 5 | 15.63 | 13 | 18.06 |

Race

| | | | | | | |
|---|---|---|---|---|---|---|
| Caucasian | 26 | 65.00 | 18 | 56.25 | 44 | 61.11 |
| Hispanic | 8 | 20.00 | 9 | 28.13 | 17 | 23.61 |
| Black | 5 | 12.50 | 3 | 9.38 | 8 | 11.11 |
| Other, | 1 | 2.50 | 2 | 6.25 | 3 | 4.17 |
| specify | | | | | | |

Foot

| | | | | | | |
|---|---|---|---|---|---|---|
| Right | 23 | 57.50 | 18 | 56.25 | 41 | 56.94 |
| Left | 17 | 42.50 | 14 | 43.75 | 31 | 43.06 |

Location

| | | | | | | |
|---|---|---|---|---|---|---|
| Toe | 13 | 32.50 | 14 | 43.75 | 27 | 37.50 |
| Heel | 18 | 45.00 | 10 | 31.25 | 28 | 38.89 |

*NS = not significant (P > 0.05)
Demographic/ulcer location variables not statistically significantly different between groups

Site audits revealed that 32 out of 72 patients (44%) had protocol violations or did not meet the criteria for participation throughout the course of the study. Of the 32 excluded patients, 24 (75%) had protocol violations and eight (25%) failed to complete treatment. The protocol violations appeared to affect outcomes; thus, the PP dataset, at audit completion, became the primary dataset for analysis—19 patients were in the PRP gel group and 21 patients were in the control group. These patient outcomes reflect patients/wounds treated PP.

In the PP group, only the proportion of Caucasian versus non-Caucasian participants was significantly different (P=0.02). The proportion of Caucasians was significantly higher in the PRP gel group. No statistically significant difference between the PRP gel group and the control group related to age, HgbA1C, wound area, wound volume, sex, or wound location were observed. This is the same as the ITT group (see Table 4).

TABLE 4
PER PROTOCOL PATIENT DEMOGRAPHICS AND
WOUND CHARACTERISTICS AT BASELINE

| | |
|---|---|
| RP Gel (n = 19) | Control (n = 21) |

| | | | | | | | | | | | |
|---|---|---|---|---|---|---|---|---|---|---|---|
| Variable | N | Mean | SD | Min | Max | N | Mean | SD | Min | Max | P value |

Patient

| | | | | | | | | | | | |
|---|---|---|---|---|---|---|---|---|---|---|---|
| Age (years) | 19 | 58.3 | 9.7 | 43.0 | 75.0 | 21 | 55.9 | 8.1 | 45.0 | 78.0 | NS* |
| Hgb A₁C | 18 | 7.8 | 1.5 | 5.5 | 11.1 | 20 | 8.1 | 1.8 | 5.0 | 11.4 | NS |

Wound

| | | | | | | | | | | | |
|---|---|---|---|---|---|---|---|---|---|---|---|
| Area (cm²) | 19 | 3.4 | 4.5 | 0.8 | 20.0 | 21 | 3.6 | 4.0 | 0.5 | 15.8 | NS |
| Volume | 19 | 0.9 | 1.3 | 0.1 | 4.7 | 21 | 1.0 | 1.4 | 0.1 | 5.4 | NS |
| (cm³) | | | | | | | | | | | |

| | Treatment | | | | | |
|---|---|---|---|---|---|---|
| | PRP Gel (n = 19)† | | Control (n = 21)† | | Total | |
| | No. of | | No. of | | No. of | |
| Characteristics | patients | Percent | patients | Percent | patients | Percent |

Sex

| | | | | | | |
|---|---|---|---|---|---|---|
| Male | 16 | 84.21 | 16 | 76.19 | 32 | 80.00 |
| Female | 3 | 15.79 | 5 | 23.81 | 8 | 20.00 |

Race

| | | | | | | |
|---|---|---|---|---|---|---|
| Caucasian | 15 | 78.95† | 9 | 42.86† | 24 | 60.00 |
| Hispanic | 4 | 21.05 | 8 | 38.10 | 12 | 30.00 |
| Black | | | 3 | 14.29 | 3 | 7.50 |
| Other, | | | 1 | 4.76 | 1 | 2.50 |
| specify | | | | | | |

Foot

| | | | | | | |
|---|---|---|---|---|---|---|
| Right | 14 | 73.68 | 10 | 47.62 | 24 | 60.00 |
| Left | 5 | 26.32 | 11 | 52.38 | 16 | 40.00 |

Location

| | | | | | | |
|---|---|---|---|---|---|---|
| Toe | 6 | 31.58 | 9 | 42.86 | 15 | 37.50 |
| Heel | 10 | 52.63 | 6 | 28.57 | 16 | 40.00 |

*NS = not significant (P > 0.05)
†P = 0.02, two-sided Fisher's Exact Test

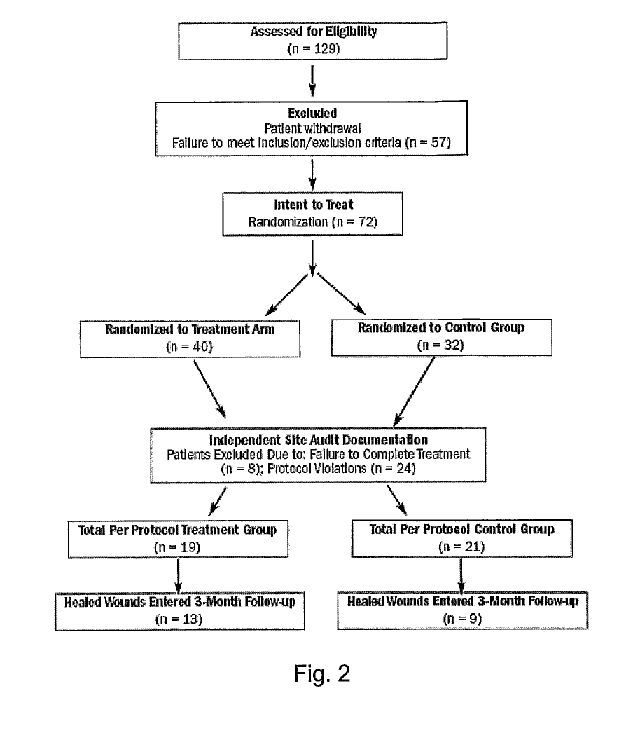

Efficacy outcomes. In the PP dataset, 13 of 19 (68.4%) patients in PRP gel and nine out of 21 (42.9%) patients in the control group healed (P=0.125, two-sided Fisher's exact test). The 95% CI for the percent proportion of completely healed wounds was 47.5% to 89.3% and 21.7% to 64.0% for PRP gel and control groups, respectively. The Kaplan-Meier median time to complete closure was 45 days for PRP gel compared to 85 days for control (log-rank test, P=0.126) (see FIG. 2).

Although the inclusion/exclusion criteria included wounds with an area range at least 0.5 cm² to no larger than 20 cm², size frequency distributions showed that the majority (35 out of 40, 88%) of wound sizes were in the range of both 7.0 cm² in area and 2.0 cm³ in volume and five (three patients/wounds in the PRP and two in the control group) were outliers. The mean area of the outliers was 10.53 cm² (SD 8.9) and 14.63 cm² (SD 1.6) for the PRP and control group, respectively. Because the size range of this group correlates with the average wound size in multiple published diabetic foot ulcer studies (Margolis 2003), the analyses were repeated using ulcers in this size range only. This subset of the PP dataset will be referred to as the majority wounds group.

When standardized for size, (mean area PRP=2.01 cm² (SD 1.3) and control 2.43 cm² (SD 1.6), the proportion of completely healed wounds was 13 out of 16 (81.3%) and eight out of 19 (42.1%) in PRP gel and control treatment groups, respectively (P=0.036, Fisher's exact test). The 95% CI for the percent proportions of completely healed wounds was 62.1% to 100% for the PRP and 19.9% to 64.3% for the control group.

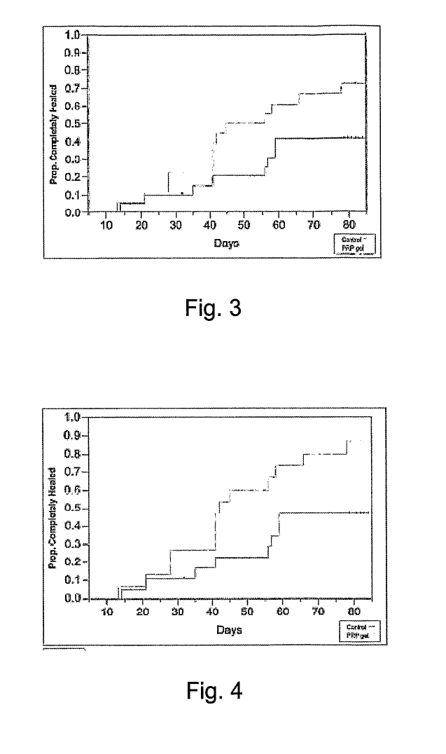

The Kaplan-Meier curves of proportion for completely healed wounds over time for the majority wound dataset showed that the two curves started separating from each other on about day 28 (see FIG. 3), log-rank test, P value=0.018.

When evaluating the range of healing outcomes in the four investigative site groups, wide variations in healing outcomes between the site groups were observed. For the PP dataset, the site group percent of complete healing proportion varied from 50% to 100% for PRP gel-treated wounds and from 25% to 67% for control-treated wounds. For the majority wound dataset, the site group percent of complete healing proportion varied from 60% to 100% and from 25% to 60% in the PRP gel- and control-treated wounds, respectively. A trend for increased wound healing in the PRP group compared to the control group also was observed. Despite the between-site group variations, healing outcomes in each treatment group were consistent and similar in both the total dataset of the per protocol and majority wound groups.

Rate of healing. In the PP dataset, the average wound area closure rate per day was 0.051 cm² for the PRP gel group versus 0.054 cm² for the control group. In the majority wound dataset, the wound area closure per day was 0.042 cm² for the PRP gel group and 0.043 cm² for the control group; these differences were not statistically significant.

In the PP dataset, wounds in the PRP gel group healed after a mean of 42.9 days (SD 18.3) compared to 47.4 days (SD 22.0) for wounds in the control group. While the number of days to healing was the same in the majority wound group (mean 42.9 and 42.8 days), 81.3% of PRP gel-treated wounds and 42.1% of control gel-treated wounds healed during that time.

Follow-up. Of the 40 patients in the PP dataset, 22 with healed wounds participated in the 12-week follow-up phase; of those, one in the PRP gel group had a wound that reopened. None of the control-treated patients' wounds re-opened; this difference was not statistically significant.

Safety outcomes: adverse events (AE). An AE in a clinical study patient who has been administered an investigational agent is any unusual medical occurrence that has appeared or worsened after the start of study whether or not the occurrence was related to the use of the investigational product. Adverse events were captured for any clinical abnormalities that appeared or worsened between the patient's start of the 7-day screening period and 30 days after receiving the last dose of study treatment.

Of the 127 adverse events, five occurred in two patients before randomization during the 7-day screening period. Of the remaining 122 adverse events occurring after randomization, 60 (49%) were in the PRP gel group and 62 (51%) in the control group. Of these, two were identified as definitely related to the treatment: one case of contact dermatitis occurred in a PRP gel treated wound and one instance of maceration occurred in a control treated wound.

Safety outcomes: serious adverse events (SAE). An SAE is an adverse event that meets any of the following outcome criteria: is fatal; is life-threatening (i.e., the patient was, in the view of the Investigator, at immediate risk of death from the reaction as it occurred; however, it does not include a reaction that, had it occurred in a more serious form, might have caused death); requires or prolongs inpatient hospitalization; results in significant or persistent disability/incapacity; is a congenital anomaly or birth defect; is an important medical event, based on appropriate medical judgment, that may jeopardize the patient or require the patient to seek medical or surgical intervention to prevent one of the other outcomes above.

Of the 122 adverse events after randomization, 23 were classified as serious adverse events; six occurred in the PRP gel group and 17 in the control group. All serious adverse events were unlikely or unrelated to device usage as defined by the investigators (see Table 5).

TABLE 5
REPORTED SERIOUS ADVERSE EVENTS (N = 23 EVENTS)

| | | | | | |
|---|---|---|---|---|---|
| | | | | Severity as | |
| | | | | determined | |
| | During | Post | | by | Relationship |
| Medra Term | Treatment | Treatment | Event | Investigator | to device |

PRP Gel Group (6 events; 5 patients)

| | | | | | |
|---|---|---|---|---|---|
| Myocardial infarction | | X | Death | Severe | Unrelated |
| Congestive heart failure | X | | Hospitalization | Mild | Unlikely |
| Pneumonia | X | | Hospitalization | Moderate | Unlikely |
| Pneumonia | X | | Hospitalization | Mild | Unrelated |
| Pneumonia; osteomyelitis | X | | Hospitalization | Mild | Unrelated |

Control Group (17 events; 7 patients)

| | | | | | |
|---|---|---|---|---|---|
| Localized infection | X | | Hospitalization | Severe | Unlikely |
| Chest pain (gallstones) | X | | Hospitalization | Mild | Unlikely |
| Infected left foot; left foot | X | | Hospitalization | Mild | Unlikely |
| ulcer | | | | | |
| Bacterial arthritis/ | X | | Hospitalization | Moderate/severe | Unlikely |
| encephalopathy | | | | | |
| Gangrene, anemia, renal | | X | Death | Severe | Unlikely |
| failure, cardio-respiratory | | | | | |
| arrest | | | | | |
| Diabetic foot, cellulitis, | X | | Hospitalization | Severe | Unlikely |
| osteomyelitis | | | | | |
| Cellulitis, arthritis | X | | Hospitalization | Mild/severe | Unrelated |
| bacterial, atrioventricular | | | | | |
| block, elevated blood | | | | | |
| glucose | | | | | |

Clinical laboratory results. To analyze the safety of treating patients with the PRP gel, laboratory tests were conducted according to the study's predetermined time-frame. Because a prospective trial on the use of PRP gel in patients with diabetes had not been conducted before, questions were raised whether the blood draws, use of bovine thrombin, or the treatment itself would have a systemic effect on persons with diabetes. These concerns determined the clinical laboratory tests that were conducted during the trial (see Table 6).

TABLE 6
CHANGES IN CLINICAL LABORATORY RESULTS

| | PRP Gel (n = 40) | | Control (n = 32) | | |
|---|---|---|---|---|---|
| Laboratory Test | Baseline | Endpoint | Baseline | Endpoint | |
| Hematology | (mean) | (mean) | (mean) | (mean) | P value* |
| Hemoglobin (G/DL) | 13.7 | 13.4 | 13.1 | 12.8 | 0.740 |
| Hematocrit (%) | 40.6 | 40.1 | 39.2 | 38.5 | 0.644 |
| Platelet count (10\hat{ }3/UL) | 264 | 280 | 263 | 262 | 0.076 |
| White blood cells (10\hat{ }3/UL) | 8.0 | 7.9 | 7.8 | 8.0 | 0.877 |

Chemistry

| | | | | | |
|---|---|---|---|---|---|
| Albumin (G/DL) | 3.8 | 3.7 | 3.7 | 3.6 | 0.4693 |
| Bicarbonate (MEQ/L) | 23.9 | 23.7 | 23.9 | 23.4 | 0.6674 |
| Blood urea nitrogen (MG/DL) | 21.1 | 19.6 | 20.7 | 23.1 | 0.0405 |
| Chloride (MEQ/L) | 101.5 | 102.2 | 102.1 | 102.4 | 0.5721 |
| Creatinine (MG/DL) | 1.1 | 1.1 | 1.1 | 1.1 | 0.4143 |
| Glucose, serum (MG/DL) | 187.2 | 202.7 | 175.6 | 211.5 | 0.4045 |
| Potassium (MEQ/L) | 4.6 | 4.5 | 4.4 | 4.5 | 0.2343 |
| Sodium (MEQ/L) | 138.0 | 137.9 | 137.8 | 137.3 | 0.6172 |
| Hemoglobin A1C (% HB) | 8.0 | 8.5 | 8.0 | 8.0 | 0.1232 |
| Factor V activity (%) | 105.2 | 104.8 | 101.0 | 103.1 | 0.6113 |

Clotting Factors

| | | | | | |
|---|---|---|---|---|---|
| PT (seconds) | 12.8 | 13.4 | 13.0 | 13.2 | 0.8545 |
| PTT (seconds) | 29.7 | 31.9 | 30.2 | 30.3 | 0.3738 |
| TT (seconds) | 12.4 | 12.9 | 12.8 | 12.4 | 0.4315 |
| TT Human (seconds) | 11.3 | 11.7 | 11.3 | 11.1 | 0.2196 |

*Wilcoxon rank sum test, two-sided

Of the 72 participating patients, 56 (78%) returned for the day 168 laboratory tests. No statistically or clinically significant differences were noted between the PRP gel and control from baseline to end point laboratory shifts in hematology, clotting factors, and Factor V tests. Although no statistically significant difference was noted between the PRP gel and control in relation to shifts of clotting factors, a shift (increase) was noted in PT and PTT results in both treatment groups. No clinically important changes in clotting factors that would cause concern about the effect of the PRP gel or control on Factor V activity were found during an independent monitor review of the medical records, including concomitant medications.

No clinical or statistically significant differences were noted in chemistry test results between the PRP gel and control from baseline to end point for sodium, potassium, chloride, bicarbonate, creatinine, or albumin. A statistically significant difference was observed between treatments in the change from baseline for Blood Urea Nitrogen (BUN); the BUN of the PRP gel-treated patients decreased while the BUN of control patients increased (no explanation was determined).

Serum glucose or HbA1C results showed that more patients shifted to high at end point in the PRP gel compared to the control group. These differences were not statistically significant or clinically meaningful; they suggested that more patients in the PRP gel group had uncontrolled diabetes.

These safety results document the minimal occurrence of adverse events. No serious adverse events were attributable to PRP gel and minimal were attributable to laboratory shifts. These effects were comparable with the control group.

### Discussion

This is the first reported prospective, randomized, blinded, controlled trial in the U.S. on the use of PRP for the treatment of diabetic foot ulcers. In this FDA-approved study, some of their requirements (certain inclusion/exclusion criteria, blinding system, choice of control treatment, extensive laboratory tests, and documentation) added to the rigor and complexity of the study design, which in turn caused some difficulty enrolling patients.

This study comprised two levels of screening: pre- and active screening. Initially, an investigator evaluated whether a patient was a potential candidate for the study. Approximately 650 patients were pre-screened (i.e., reviewed by the investigator for possible inclusion) to secure the 129 active screening patients. Active screening comprised baseline wound assessment, physical exam, laboratory tests, wound culture, vascular tests, wound excision/debridement, and the 7-day screening period for patients meeting the requirements. From the 129 actively screened patients, 72 were randomized and 40 met study protocol requirements (the clinical data audit prompted exclusion of 32 patients, dropping the PP number to 40 patients). Thus, ultimately, only 6% of patients who participated in the pre-screening process were enrolled.

During the analysis of the PP group, frequency distribution demonstrated that the majority of the wounds (35 out of 40) randomized into the study met the criteria of wound area ≦7.0 cm² and volume ≦2.0 cm³. The remaining five larger wounds had areas of 9 cm² to 20 cm² and results of various studies suggest that a wound size of <7.0 cm² is most common. (Margolis et al. 1999; Lee 1980; Kalbfleisch et al. 1980.) Efficacy variables also were analyzed for the subset of “majority wound.”

Average baseline area in the majority wound group was similar to that reported in a tissue-engineered product study (n=208: mean wound area 2.97-3.1 cm²) (Veves et al. 2001), another tissue-engineered product (n=15 wounds; efficacy noted in wounds <6 cm²) (Lipkin et al. 2003), two recombinant growth factor studies (n=118 patients and 132 wounds respectively; mean area 5.5 cm² and 2.6 cm²) (Steed 1995; Wieman et al. 1998), and one retrospective study of diabetic foot ulcers (n=26,599 patients of which 5,320 wounds averaged 1.53 cm², 5,320 wounds averaged 1.84 cm², and 5,319 wounds averaged 4.41 cm² in area (Margolis 2001). In the largest study published to date, 60% of patients had wounds that matched the majority group in this PRP gel study, increasing the potential external validity of the current study results. (Margolis 2001.)

In the majority wound group, PRP gel-treated wounds were significantly more likely to heal than control-treated wounds even though healing rates in the control group were higher (42% healed after 12 weeks) than most control group healing rates reported in other studies. (Margolis 1999.) Specifically, results of a meta-analysis of healing outcomes in the control arm of other diabetic foot prospective studies suggest that 24% can be expected to heal after 12 weeks of providing good care. (Id.) Most studies use wet-to-moist gauze saline gauze dressings, the recognized standard treatment by the medical community (American Diabetes Association Jan. 25, 2006) as a control dressing. However, wet-to-moist dressings require dressing changes three to four times daily by the patient or caregiver and may not provide an optimal moist environment for healing. Some studies have shown that hydrocolloid dressings may be more effective than wet-to-moist for the treatment of certain types of ulcers. (Bradley et al. 1999.)

The power calculations were based on reported results of control treatments in other studies and a sample size of 27 in each treatment arm. The better-than-expected control healing rates and large number of protocol violations caused underpowering of the PP and the majority wound groups yet some statistically significant differences were observed, suggesting that larger between-group differences could be expected in studies using a larger sample size.

The FDA's concern about the effects of frequent though small amount of blood collection (30 mL or less each visit) on health and safety as patients underwent periodic hematology and other laboratory tests throughout the trial was addressed. Test results documented that these frequent blood draws did not reduce the hemoglobin, hematocrit, or platelet count. Because bovine thrombin was used in the processing to activate the PRP and evidence exists of Factor V leading to bleeding disorders in patients exposed to large doses of bovine thrombin (Margolis 2003), concerns were raised as to whether patients would similarly develop Factor V antibodies to the bovine thrombin with small frequent exposure to it. To evaluate this potential impact, Factor V tests occurred at 6-week intervals for all patients regardless of randomization arm and at 24 weeks post randomization. None of the patients demonstrated any Factor V inhibition throughout the study and into the follow-up. This is the first time that this has been documented in a prospective study.

Laboratory tests indicated that the majority of patients had elevated blood glucose throughout the study period. Elevated glucose levels have been documented to reduce healing (Jeffcoate et al. 2004; Marston 2006); however, in this study, the majority of the wounds healed.

Numerous articles have been published regarding the use of PRP in the surgical setting; notably, the orthopedic, plastic surgery, and dental field. (Kassolis et al. 2005; Grageda 2004; Weilbrich et al. 2004.) Some of the PRP separation systems used require specialized technicians to perform the necessary procedures. This is the first study of an autologous PRP separation system to heal wounds that can be used by health professionals within a traditional healthcare setting. A small, compact, point-of-care system such as the one described herein makes this technology available to multiple care providers, including physician offices, hospital units, outpatient clinics, long-term care facilities, and home health care staff.

### CONCLUSION

We have determined that PRP gel is safe for use in the treatment of nonhealing diabetic foot ulcers. In the most common size of diabetic foot ulcers (≦7.0 cm² in area and ≦2.0 cm³ in volume), PRP gel-treated wounds are also significantly more likely to heal than control gel treated wounds. Treating wounds with PRP or saline gel resulted in healing in approximately 6 weeks, but in the most common wound sizes, almost twice as many PRP treated wounds healed in that time-frame. The number of adverse events was minimal; no adverse events were serious. Further, the study demonstrated that bovine thrombin used in the preparation of PRP does not cause Factor V inhibition; thus, it does not cause coagulopathy. In addition, withdrawal of a small amount of blood twice weekly did not affect patient hemoglobin, hematocrit, or platelet count. Clinically meaningful shifts in laboratory values studied from baseline to end point were not observed. This type of PRP system could be utilized by healthcare providers to treat diabetic foot ulcers in multiple settings. Using PRP gel to treat diabetic foot ulcers may not only enhance healing, but it also may prevent lower extremity amputations caused by nonhealing wounds.

Implications for future research include implementing a trial design that would permit greater subject enrollment on a larger sample size to validate these results. This trial would not need to re-evaluate some of the major questions answered in this trial, such as Factor V inhibition, impact on the patient's hematology and other clinical laboratory outcomes, and impact of a control that had not performed in a prospective trial previously. Diabetic foot ulcers with challenging presentations (i.e., mild to moderate vascular disease, exposed tendon or bone, patient hyperglycemia, and/or inadequate nutritional status) could be studied to determine whether PRP gel could assist in healing in these compromised scenarios. In addition, studies to confirm that this novel therapy is synergistic with other advanced wound care modalities could be conducted.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A method of treating a wound of a patient comprising:
withdrawing a predetermined amount of whole blood from the patient;
separating the whole blood into red blood cells, white blood cells and platelet-rich plasma;
extracting the separated platelet-rich plasma;
contacting an effective amount of ascorbic acid to the platelet-rich plasma;
contacting an effective amount of calcified thrombin to the extracted platelet-rich plasma to activate the platelets and to obtain a gel; and
contacting an effective amount of the gel to the wound.

2. The method of claim 1 wherein the step of withdrawing a predetermined amount of whole blood from the patient comprises withdrawing the predetermined amount of whole blood from the patient into one or more blood collection tubes configured to be spun in a centrifuge at a point-of-care.

3. The method of claim 2 wherein the step of separating the whole blood into red blood cells, white blood cells, and platelet-rich plasma comprises spinning the one or more blood collection tubes in a centrifuge configured to be operated at the point-of-care.

4. The method of claim 3 wherein the step of spinning the one or more blood collection tubes in a centrifuge configured to be operated at the point-of-care comprises spinning the one or more blood collection tubes in the centrifuge for about 1.5 minutes at the point-of-care.

5. The method of claim 4 wherein the step of extracting the separated platelet-rich plasma comprises extracting the separated platelet-rich plasma into a syringe.

6. The method of claim 5 wherein the step of contacting an effective amount of ascorbic acid to the platelet-rich plasma comprises adding the ascorbic acid into the syringe and inverting the syringe to mix.

7. The method of claim 6 wherein the step of contacting an effective amount of calcified thrombin to the extracted platelet-rich plasma to activate the platelets and to obtain a gel comprises adding the calcified thrombin into the syringe and inverting the syringe several times to mix.

8. The method of claim 1 wherein the step of withdrawing a predetermined amount of whole blood from the patient comprises withdrawing about 5 to about 30 cc of whole blood from the patient.

9. The method of claim 8 wherein the step of withdrawing a predetermined amount of whole blood from the patient comprises withdrawing about 20 cc of whole blood from the patient.

10. The method of claim 8 wherein the step of extracting the separated platelet-rich plasma comprises extracting about 8 mL of the separated platelet rich plasma.

11. The method of claim 10 wherein the step of contacting an effective amount of ascorbic acid to the platelet-rich plasma comprises contacting about 1 mL of ascorbic acid to the platelet-rich plasma.

12. The method of claim 11 wherein the step of contacting an effective amount of calcified thrombin to the extracted platelet-rich plasma to activate the platelets and to obtain a gel comprises contacting about 1 mL of calcified thrombin to the extracted platelet-rich plasma.

13. A system for making a composition for treating a wound of a patient at a point-of-care comprising:
a blood-drawing device for drawing a predetermined amount of whole blood from the patient into one or more blood collection tubes;
one or more blood collection tubes configured to be spun in a centrifuge;
a centrifuge configured to operate at the point-of-care to separate the whole blood into red blood cells, white blood cells and platelet-rich plasma;
an extracting device for extracting a predetermined amount of the separated platelet-rich plasma from the one or more blood collection tubes; and
a mixing device for coupling to the extracting device and adding a predetermined amount of one or more reagents to the separated platelet rich plasma.
